(19) United States
(12) Patent Application Publication
(10) Pub. No.: US 2014/0179740 A1
(43) Pub. Date: Jun. 26, 2014

(54) STABLE LIQUID REMIFENTANIL FORMULATIONS

(71) Applicant: MYLAN INC., Morgantown, WV (US)

(72) Inventors: Daniel Swaim ROBINS, Oak Park, IL (US); Leena Selvaraj, Solon, OH (US); Rajaram Vaidyanathan, Irvine, CA (US)

(73) Assignee: Mylan Inc., Morgantown, WV (US)

(21) Appl. No.: 13/724,711

(22) Filed: Dec. 21, 2012

(51) Int. Cl. A61K 31/4468; A61K 9/00

(52) U.S. Cl. CPC A61K 31/4468; A61K 9/0019
USPC 514/329

(57) ABSTRACT

A stable, non-aqueous, liquid pharmaceutical composition contains remifentanil dissolved in a non-aqueous solvent. The formulation is provided with a pH ranging from about 2.0 to about 3.5 and can be used as an injectable. A method for preparing the formulation is also provided.

### FIELD

This invention relates to stable liquid remifentanil formulations which are suitable for injection of a patient.

### BACKGROUND

The present invention relates to stable liquid injectable formulations of remifentanil, i.e., 3-[4-methoxycarbonyl-4-[(1-oxopropyl)phenylamino]-1-piperidine] propanoic acid, methyl ester.

U.S. Pat. No. 5,019,583 to Feldman, et al. discloses a specific class of N-phenyl-N-(4-piperidinyl)amides and, particularly, methyl[4-(methoxycarbonyl)-4-[(1-oxopropyl)phenylamino]-1-piperidine]-propanoate, monohydrochloride (now known as remifentanil), a μ-opioid anesthetic. These amides are potent analgesics characterized by rapid onset and an exceptionally short terminal half-life attributed to their rapid enzymatic metabolism in the blood as contrasted with the slower metabolism of other opioids in the liver. The ester linkage in the pendant chain from the piperidine nitrogen renders the amides susceptible to inactivation by plasma and tissue esterases. The drug substance contains two alkyl esters, one sterically hindered and the other sterically unhindered and susceptible to hydrolysis. The sterically unhindered ester grouping is susceptible to hydrolysis by aqueous hydrolysis and by esterases in blood and tissues. The major hydrolysis product is a monocarboxylic acid remifentanil derivative, which is significantly less potent than remifentanil. Consequently, remifentanil exhibits a rapid onset of action and rapid recovery. Rapid inactivation of remifentanil by hydrolysis provides the following advantages in anesthesia: 1) an ultra-short duration of action; 2) less variability in recovery rate even with differences in hepatic function; 3) no accumulation of drug during repeated bolus doses or infusion; and 4) more rapid recovery.

U.S. Pat. No. 5,019,583 to Feldman et al. (col. 20, line 63 to col. 21, line 23), prepares remifentanil formulations by providing “an effective analgesic amount of the particular compound, in base or acid-addition salt form, as the active ingredient . . . combined in intimate admixture with a pharmaceutically acceptable carrier, which carrier may take a wide variety of forms depending on the form of preparation desired for administration.” The reference discloses unitary dosage forms for administration orally, transdermally, rectally or parenterally. For oral liquid dosage forms, “water, glycols, oils, alcohols and the like” can be used. Parenteral compositions contain a carrier that “will usually comprise sterile water, at least in large part, though other ingredients, for example, to aid solubility, may be included.” Injectable solutions are taught wherein “the carrier comprises isotonic saline solution, glucose solution or a mixture of saline and glucose solution [and] [i]njectable suspensions may also be prepared in which case appropriate liquid carriers, suspending agents and the like may be employed.”

U.S. Pat. No. 5,466,700 to Batenhorst et al., incorporated herein by reference in its entirety, discloses methods of administering remifentanil formulations to induce anaesthesia.

U.S. Pat. No. 5,866,591 to Gatlin et al. discloses a glycine lyophilized dosage form of remifentanil hydrochloride which can be stored at 40° C. for one month with only 1% loss of active drug substance as analyzed by reverse phase high performance liquid chromatography (Ex. 16). Large batch (110 liters and 75 liters) preparations of 1 mg remifentanil hydrochloride lyophilized dosage forms were made at concentrations of 1.0 mg/mL, 2.0 mg/mL, and 5.0 mg/mL. Stability studies showed that these formulations, reconstituted with an aqueous solvent, maintained a room temperature shelf life stability of two years (i.e. the drug substance remifentanil hydrochloride is degraded by hydrolysis to an extent of less than about 10% in two years). The '591 patent does not list non-aqueous organic solvents as re-suspension agents.

U.S. Pat. No. 6,660,715 (“the '715 patent”) teaches a formulation for pulmonary delivery of bioactive agents such as fentanyl which are delivered in biocompatible organic solvents suitable for aerosolization. The bioactive agents are dissolved or dispersed in biocompatible organic solvents described as non-aqueous. The '715 patent thus teaches a non-aqueous, liquid system for delivering fentanyl to the lung via aerosolization, but does not disclose non-aqueous remifentanil for intravenous administration.

U.S. Publication No. 2007/0261695 (“the '695 publication”) discloses a non-aqueous, liquid fentanyl formulation for sublingual administration, wherein fentanyl is dissolved in a non-aqueous, organic solvent which includes, inter alia, ethanol; ethylene glycol; polyethylene glycol. However, no mention is made of using the non-aqueous, liquid fentanyl formulation for intravenous administration, nor is any mention made of remifentanil compositions.

U.S. Publication No. 2008/0317828 (“the '828 publication”) teaches an edible oral mucosal patch comprising an opioid (e.g. remifentanil) which is dissolved in a non-aqueous solvent (e.g. ethanol). The '828 publication does not disclose a liquid, non-aqueous preparation for intravenous administration.

U.S. Publication No. 2009/0176834 (“the '834 publication”) discloses a liquid fentanyl spray for sublingual administration. The '834 publication discloses a number of solvents to dissolve fentanyl which include, inter alia, water, lower alcohols and polyethylene glycols. The '834 publication does not teach remifentanil, nor does the '834 publication appreciate the increased potential for hydrolysis in an aqueous solvent system.

WO2008135263 (“the '263 publication”) discloses a transdermal therapeutic system, including remifentanil, in a reservoir containing an organic solvent such as ethanol.

The hydrochloride salt of remifentanil is commercially available for use as an injectable aqueous formulation in anesthesia under the tradename ULTIVA®. This formulation is provided as a lyophilized solid and contains glycine. This formulation must be combined with sterile water or other aqueous IV fluids shortly before use to provide the injectable formulation. The injection solution must be used within twenty four hours after mixing with sterile water and most other IV fluids to avoid breakdown of the remifentanil into by-products of ester hydrolysis. Moreover, the presence of glycine contraindicates the use of ULTIVA® for epidural or intrathecal administration, according to its FDA-approved label.

It would be desirable to provide an injectable liquid formulation of remifentanil which is less susceptible to breakdown into by-products of ester hydrolysis. It would also be desirable to provide an injectable formulation of remifentamil which does not require reconstitution by dilution with liquid shortly before use and which can be stored for significant periods at room temperature or under refrigeration. Moreover, it would be advantageous to provide an injectable liquid formulation of remifentanil which can be used for epidural or intrathecal administration.

### SUMMARY

In one aspect, the present disclosure relates to an injectable liquid pharmaceutical composition comprising remifentanil dissolved in a non-aqueous solvent containing at least one hydrophilic organic component, wherein the composition is water-miscible and has a pH ranging from about 2.0 to about 3.5 and at least one of: i) a room temperature shelf life stability of at least about one year and ii) a refrigerated shelf life stability at about 5° C. of at least about two years.

In certain embodiments of this aspect of the invention, the at least one hydrophilic organic component of the composition is a water miscible hydrophilic organic component.

In another embodiment, the non-aqueous solvent of the composition contains at least one of glycol and polyethylene glycol.

In still other embodiments, the non-aqueous solvent of the composition contains a mixture of glycol and polyethylene glycol in amounts ranging from about 0.1 volumes to about 10 volumes glycol per volume of polyethylene glycol. In yet other embodiments, the non-aqueous solvent of the composition contains a mixture of glycol and polyethylene glycol added in amounts ranging from about 1 volume to about 8 volumes glycol per volume of polyethylene glycol.

In still yet other embodiments, the non-aqueous solvent of the composition contains a mixture of pharmaceutically acceptable glycol and polyethylene glycol containing amounts ranging from about 2 volumes to about 6 volumes glycol per volume of polyethylene glycol. The glycol can be selected from propylene glycol (1,2-propanediol), glycerol or any other pharmaceutically acceptable glycol. The polyethylene glycol can be selected from PEG-200, PEG-300, PEG-400 and PEG-500 and PEG-600, with PEG-400 being especially preferred.

In yet still other embodiments, the non-aqueous solvent of the composition contains a mixture of glycol and polyethylene glycol in amounts ranging from about 3 to about 5 volumes of glycol per volume of polyethylene glycol, the glycol is propylene glycol, and the polyethylene glycol is PEG-400.

In another embodiment, the non-aqueous solvent contains a mixture of glycol and polyethylene glycol in amounts of about 4 volumes of glycol per volume of polyethylene glycol and the pH of the composition ranges from about 2.0 to about 3.0.

In yet other embodiments, the composition is substantially free of amino acid, e.g., substantially free of glycine.

In some embodiments, the remifentanil content after about 7 months at about 40° C. is greater than about 91% of initial remifentanil content, e.g., greater than about 93% of initial remifentanil content, typically, greater than about 95% of initial remifentanil content.

In various embodiments, the composition of this aspect of the disclosure comprises no more than about 10 wt. % total impurities for 1 mg doses, no more than about 8 wt. % total impurities for 2 mg doses, and no more than about 6 wt. % total impurities for 5 mg doses.

In some embodiments, the impurities comprise at least one of hydrolysis degradation product GR90291X and heat degradation product GR94219X.

In certain embodiments, the composition of this aspect of the disclosure is suitable for parenteral administration in a patient.

In some embodiments, the composition of this aspect of the disclosure is suitable for intravenous administration in a patient.

In other embodiments, the remifentanil in the composition is in the form of a free base.

In still other embodiments, the remifentanil in the composition is in the form of a salt, e.g., a hydrochloride salt.

In some embodiments, the composition is sterilized.

In yet other embodiments, the composition of this aspect of the disclosure contains remifentanil hydrochloride in the range of about 0.1 mg/mL to about 5 mg/mL, e.g., in an amount of about 1 mg/mL.

In still other embodiments, the composition is provided in a single unit dosage amount selected from 0.25 mg, 0.5 mg, 1 mg, 2 mg, and 5 mg of remifentanil.

In certain embodiments of this aspect of the disclosure, the disclosure relates to a method of preparing an infusion mixture which comprises adding an aqueous solution to the injectable liquid pharmaceutical composition to provide an infusion mixture containing from about 20 to about 250 μg/mL of remifentanil.

In other embodiments of this aspect of the disclosure, the disclosure relates to an infusion mixture which is prepared by adding an aqueous solution to the injectable liquid pharmaceutical composition to provide an infusion mixture containing from about 20 to about 250 μg/mL of remifentanil.

In another aspect, the present disclosure relates to a method for preparing an injectable liquid composition of at least one of: i) a room temperature shelf life stability of at least about one year and ii) a refrigerated shelf life stability at about 5° C. of at least about two years, comprising mixing remifentanil with a non-aqueous solvent containing at least one hydrophilic organic component, and adjusting the pH of the resulting mixture to a pH ranging from about 2.0 to about 3.5.

In various embodiments of this aspect, the mixing comprises sonication.

In another aspect, the present disclosure is related to a method of providing analgesia in a mammal comprising administering to such mammal an analgesically effective amount of an injectable liquid pharmaceutical composition comprising remifentanil dissolved in a non-aqueous solvent containing at least one hydrophilic organic component, wherein the composition is water-miscible, has a pH ranging from about 2.0 to about 3.5 and at least one of: i) a room temperature shelf life stability of at least about one year and ii) a refrigerated shelf life stability at about 5° C. of at least about two years.

In still another aspect, the present disclosure relates to an injectable liquid pharmaceutical composition comprising remifentanil hydrochloride dissolved in a non-aqueous solvent containing propylene glycol and polyethylene glycol, wherein the composition has a pH ranging from about 2.0 to about 3.5 and at least one of: i) a room temperature shelf life stability of at least about one year and ii) a refrigerated shelf life stability at about 5° C. of at least about two years.

In some embodiments of this aspect of the disclosure, the composition contains remifentanil hydrochloride in an amount ranging from about 0.5 to about 2 mg/mL.

In certain embodiments of this aspect of the disclosure, the composition is provided in a single unit dosage amount selected from 0.25 mg, 0.5 mg, 1 mg, 2 mg, and 5 mg of remifentanil.

In various embodiments of this aspect of the disclosure, the non-aqueous solvent of the composition contains a mixture of propylene glycol and polyethylene glycol in amounts ranging from about 3 to about 5 volumes of glycol per volume of polyethylene glycol, and the polyethylene glycol is PEG-400.

In still other embodiments of this aspect of the disclosure, the non-aqueous solvent of the composition contains a mixture of propylene glycol and polyethylene glycol in amounts of about 4 volumes of propylene glycol per volume of polyethylene glycol, and the pH is in a range of from about 2.0 to about 3.0.

In yet another embodiment, the present disclosure relates to an injectable liquid pharmaceutical composition comprising remifentanil dissolved in a non-aqueous solvent containing at least one hydrophilic organic component, wherein the composition is water-miscible, has a pH ranging from about 2.0 to about 3.5 and a shelf life stability at about 40° C. of at least about six months.

### DETAILED DESCRIPTION

The present disclosure provides an injectable liquid formulation of remifentanil. More particularly, the disclosure provides an injectable, liquid, storage-stable remifentanil formulation containing a non-aqueous solvent having at least one hydrophilic organic component.

Remifentanil generally exists in one of two forms, unprotonated (the free base), or protonated and accompanied by an appropriate counter ion, for example chloride, as in remifentanil hydrochloride. As used herein, “remifentanil” can refer to either form. When a specific form is intended, “remifentanil” will be modified appropriately as either the free base or as whichever particular salt is referred to, for example remifentanil hydrochloride.

Remifentanil hydrochloride is a μ-opioid agonist chemically designated as a 3-[4-methoxycarbonyl-4-[(1-oxopropyl) phenylamino]-1-piperidine]propanoic acid methyl ester, hydrochloride salt, C₂₀H₂₈N₂O₅.HCl, with a molecular weight of 412.91. It has the following chemical structure I:

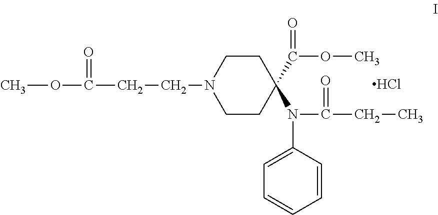

For present purposes, an injectable liquid remifentanil formulation is a formulation suited to parenteral administration by injection, e.g., intravenous, intra-arterial, intra-muscular, and subcutaneous administration, with intravenous administration being preferred. A liquid formulation is one that is normally liquid at room temperature conditions.

Storage-stable remifentanil formulations, for purposes of this disclosure, are those which exhibit minimal degradation of remifentanil, e.g., less than about 10 wt. % remifentanil, e.g., less than about 5 wt. %, or even less than about 1 wt. %, over an extended storage period. The extended storage can be under room temperature conditions for at least six months in some embodiments, or at least one year in certain embodiments, or for at least two years in other embodiments. For present purposes, room temperature conditions can include temperatures ranging from about 18° C. (64° F.) to about 24° C. (75° F.), with a temperature of about 21° C. (70° F.) being typical. Alternatively, the extended storage can be under refrigeration conditions, i.e., above 0° C. and below about 8° C. (46° F.), typically about 5° C. (41° F.), for at least six months in some embodiments, or for at least one year in certain embodiments, or for at least two years in other embodiments, or even longer, e.g., at least three years.

Regulatory specifications for shelf-life storage is for “total impurities” (defined as the percentage of remifentanil-derived degradation products present after storage relative to the amount of remifentanil measured prior to storage) of not more than 6.0%, 8.0%, and 10.0% by weight for 5 mg, 2 mg, and 1 mg doses, respectively.

Major degradation products of remifentanil are: GR90291X, a hydrolysis degradant, GR94219X, a secondary amine, and “RRT 0.9 impurity,” a transester. Certain embodiments of the injectable liquid formulation of the disclosure, contain no more than about 10 wt. % remifentanil-derived degradation products, no more than about 5 wt. % remifentanil-derived degradation products, no more than about 2 wt. % remifentanil-derived degradation products, no more than about 1 wt. % remifentanil-derived degradation products, no more than about 0.5 wt. % remifentanil-derived degradation products, or no more than about 0.1 wt. % remifentanil-derived degradation products.

Hydrolysis degradant GR90291 has a molecular weight of 362.42 g/mol and is also known as GI90291, GI-90291, GR90291c, as well as 4-Methoxycarbonyl-4-((1-oxopropyl)phenylamino)-1-piperidine propionic acid and 1-Piperidinepropanoic acid, 4-(methoxycarbonyl)-4-((1-oxopropyl)phenylamino), and is assigned CAS No. 132875-68-4. GR90291 has the structure II set out below:

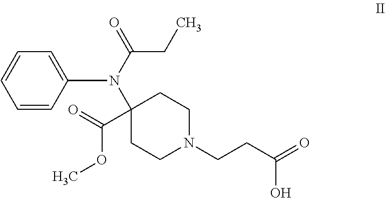 [SMILES: CCC(=O)N(c1ccccc1)C1(C(=O)OC)CCN(CCC(=O)O)CC1]

GR94219X, a secondary amine has a molecular weight of 252 g/mol and is also known as 4-Methoxycarbonyl-4-((1-oxopropyl)phenylamino)-1-piperidine propionic acid. GR94219X has the formula Structure III set out below:

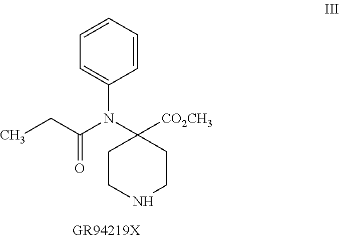 [SMILES: CCC(=O)N(c1ccccc1)C1(C(=O)OC)CCNCC1]

RRT 0.9 Impurity, is believed to result from the transesterification of remifentanil with propylene glycol (1,2-propanediol). The transesterification product, has at least one of the following structures IV and V:

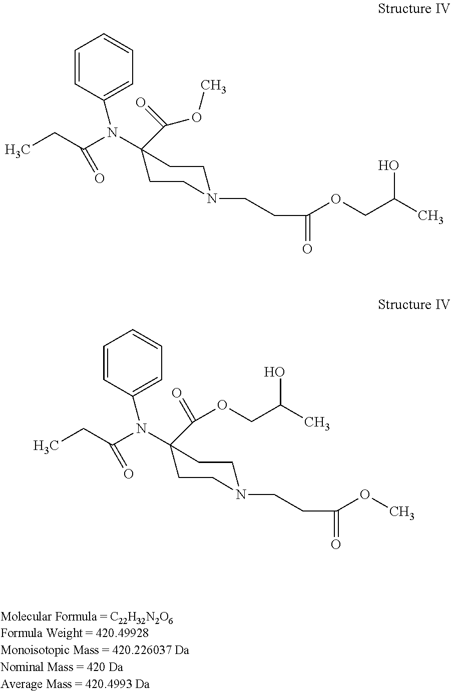

Water is substantially excluded from the formulation in order to avoid ester hydrolysis of remifentanil. The formulation contains substantially no water. Thus, water can be present only in amounts which cause no measurable degradation or minimal degradation of remifentanil during storage of the liquid formulation for at least one of: i) a room temperature shelf life stability of at least about one year and ii) a refrigerated shelf life stability at about 5° C. of at least about two years. In some embodiments, the formulation contains no more than about 2.5 wt. % water, or even no more than about 2.0 wt. % water, or even no more than about 1.8 wt. % water or even no more than about 1.0 wt. % water, or even no more than about 0.5 wt. % water, or even no more than about 0.1 wt. % water, or even no more than about 0.01 wt. % water.

In various embodiments of the disclosure, the injectable liquid remifentanil formulation is clear and colorless and contains remifentanil hydrochloride (HCl) equivalent to 1 mg/mL of remifentanil base. The pH of the injectable liquid remifentanil formulation ranges from about 2.0 to about 3.5. Remifentanil HCl has a pKa of 7.07.

In certain embodiments of the disclosure, the injectable liquid remifentanil formulation can be diluted from concentrations of about 1 mg/mL to concentrations of 20 to 250 mcg/mL with an aqueous solution, e.g., saline or Lactated Ringer's Injection, USP. In some embodiments, the injectable liquid remifentanil formulation of the disclosure is compatible with IV fluids when co-administered into a running IV administration set.

The non-aqueous solvent of the injectable liquid remifentanil formulation is a liquid which is capable of effectively dissolving remifentanil in amounts sufficient to provide a liquid containing up to 5 mg/mL remifentanil, e.g., up to about 2, up to about 1, or even up to about 0.5 mg/mL remifentanil. The solvent contains no intentionally added water.

The non-aqueous solvent comprises at least one hydrophilic organic component. Such a component is one which contains a molecule that is attracted to, and tends to be dissolved by, water. A hydrophilic molecule or portion of a molecule is one that has a tendency to interact with or be dissolved by water and other polar substances. An approximate rule of thumb for hydrophilicity of organic compounds is that solubility of a molecule in water is more than 1 mass % if there is at least one neutral hydrophile group per 5 carbons, or at least one electrically charged hydrophile group per 7 carbons.

For present purposes, suitable hydrophilic organic components include liquid alkylene glycols, polyethylene glycols, benzyl alcohol, alkyl alcohols, and glycerol.

Advantageously, the hydrophilic organic component is water-miscible. For purposes of the present disclosure, a water-miscible liquid organic component is capable of mixing in all proportions with water to form a homogeneous solution. In organic compounds the weight percent of hydrocarbon chain often determines the compound's miscibility with water. For example, among the alcohols, ethanol has two carbon atoms and is miscible with water, whereas octanol with a C₈H₁₇ substituent is not. Analogous situations occur for other functional groups. Acetic acid (CH₃COOH) is miscible with water, whereas valeric acid (C₄H₉COOH) is not. Simple aldehydes and ketones tend to be miscible with water, because a hydrogen bond can form between the hydrogen atom of a water molecule and the unbonded (lone) pair of electrons on the carbonyl oxygen atom.

When discussing the pH of the liquid compositions herein, we are referring to the pH of an aqueous or non-aqueous solution resulting from dissolving the solid composition, e.g., a lyophilized composition, in an amount of aqueous liquid or non-aqueous liquid suitable for parenteral or intravenous administration to a patient.

When discussing the pH of solid compositions, we are referring to the pH of an aqueous or non-aqueous solution resulting from dissolving the solid composition in an amount of aqueous liquid suitable for parenteral or intravenous administration to a patient. The present disclosure provides a liquid composition comprising remifentanil that maintains a room temperature shelf life stability of two years (i.e., degrades to an extent of less than 10% in two years).

In some embodiments, the compositions of this invention comprise remifentanil and at least one of the following agents: ascorbic acid, citric acid, maleic acid, phosphoric acid, hydrochloric acid, succinic acid, or tartaric acid. These agents are believed to be useful as buffering or visualization agents. In certain embodiments, the compositions contain hydrochloric acid. In some cases it may be beneficial to also have some sodium chloride, mannitol, polyvinylpyrrolidone, or other ingredients in the compositions. The injectable formulations of this invention comprise remifentanil, in amounts of at least 2 wt. %, more preferably in amounts of at least 5 wt. % of the injectable formulation. Preferably the compositions of this invention contain less than 1 wt. % of water.

Typically, no antioxidants or preservatives are added to the injectable liquid formulation of the present disclosure. However, in some instances, preservatives which inhibit or prevent microbial growth, e.g., ascorbyl palmitate, benzoic acid, butylated hydroxyl anisole, butylated hydroxytoluene, ethylenediamine, glycerin, monothioglycerol, potassium sorbate, sodium bisulfate, sodium formaldehyde sulfoxylate, sodium metabisulfite, and/or sorbic acid can be added in small amounts, if desired. In particular, a preservative can be included in the formulation where the formulation undergoes terminal steam autoclaving.

The compositions of this disclosure provide stable injectable liquid formulations of remifentanil and they may also be used to reduce the pH of typical infusion solutions. The compositions of this disclosure are buffered to a pH range of about 2.0 to about 3.5, or in some embodiments to a pH range of about 2.5 to about 3.5, or even a pH range of about 2.8 to about 3.2. Such buffering gives a solution in the stated pH ranges when dissolved in an amount of water to give a composition suitable for intravenous administration to a patient.

Typically, the undiluted liquid solutions of this disclosure that are suitable for administration, e.g., by bolus injection, can contain about 0.25 mg per mL, about 0.50 mg per mL solution, or about 1 mg remifentanil per mL solution. The injectable liquid formulations of the disclosure can be further diluted with water, isotonic saline or some other suitable IV fluid diluent to provide a mixture, suitable for infusion to a patient, which contains 1 mg remifentanil per 10 to 1,000 mL of solution. Preferred IV fluids are those that do not adversely affect the buffer. Ringer's lactate solution is not preferred. The injectable liquid compositions of this disclosure may be prepared from lyophilized compositions or they may simply be prepared from solid remifentanil.

The excipient to drug ratio in some embodiments of this invention are preferably no greater than about 75 to 1 by weight, more preferably no greater than about 40 to 1 by weight. The word “excipient” is meant to include all components other than the active drug substance. Once the injectable liquid formulations of the disclosure have been prepared, they can be placed in appropriate size vials, say, with 0.25 mg remifentanil in a 2 mL vial, 0.5 mg remifentanil in a 2 mL vial, 1 mg remifentanil in a 3 mL vial, 2 mg in a 5 mL vial, and 5 mg in a 10 mL vial. The vials can be sealed with a suitable removable stopper, e.g., a 13 mm. West 4416/50, gray, (bromobutyl) stopper, available from West Pharmaceuticals Services, Inc. of Exton, Pa., USA. Intravenous admixtures are typically prepared from the liquid formulation having 1 mg of remifentanil activity per 1 mL by diluting to a recommended final concentration of 20, 25, 50, or 250 mcg/mL prior to administration.

The compositions of this invention can be sterilized by any suitable techniques which do not substantially effect hydrolysis of the ester linkage of remifentanil, e.g., by conventional autoclaving.

The disclosure will now be described in more detail by the following non-limiting EXAMPLES. The EXAMPLES are presented to illustrate but a few embodiments of the disclosure. All parts are by weight unless otherwise indicated.

### Example 1

### Stability Study of Reconstituted ULTIVA® in Water

One vial of ULTIVA® (Lot #61235 DD; 5 mg per vial; Lyophilized product) containing 5 mg of active ingredient was reconstituted with 5.0 mL of water. The reconstituted solution was diluted with mobile phase to provide an analytical solution of 0.5 mg/mL in pH 2.5 buffer.

The stability of the analytical solution for ULTIVA® was determined using a test method wherein the mobile phase was a mixture of acetonitrile and 0.025 M Ammonium phosphate buffer at pH 2.5 (22:78, v/v). The method parameters involve a C18-HS, 250×4.6 mm I.D stainless steel column packed with Alltech Adsorbosphere, 5 μm particles; Detector Wavelength: 210 nm; column temperature: ambient; flow rate: 1.0 mL/minute (isocratic mode); injection volume: 20 μL; concentration of remifentanil: 0.5 mg/mL; and run time: 50 minutes.

The method verification was performed by analyzing Remifentanil in reconstituted ULTIVA®. The column used was a C18-HS Alltech Adsorbosphere column. The retention time of remifentanil was 13.278 minutes and the tailing factor was 3.3. There were four impurities peaks observed at relative retention time (RRT) of 0.22, 0.7 (0.67), 0.8 (0.76), and 1.4 (1.35) with respect to the retention time of remifentanil. The testing was repeated with two additional columns, Luna and Gemini, to improve the tailing factor for remifentanil. The tailing factor for Luna column was 4.1 and the tailing factor for Gemini column was 1.7. Based on the tailing factor and resolution factor results, it was decided to use Gemini column for the testing. The chromatographic elution profile for the Resolution Check Solution of Remifentanil with Gemini column is provided in TABLE 1.

TABLE 1
CHROMATOGRAPHIC ELUTION PROFILE
OF RESOLUTION CHECK SOLUTION FOR
REMIFENTANIL WITH GEMINI COLUMN

| Peak No. | RT (min) | Peak Name |
|---|---|---|
| Peak 1 | 6.344 | GI114559X |
| Peak 2 | 7.031 | GR94219X |
| Peak 3 | 7.972 | GR90291X |
| Peak 4 | 9.736 | Remifentanil |
| Peak 5 | 11.975 | GR187657X |
| Peak 6 | 15.890 | GI114560X |
| Peak 7 | 18.099 | GR151503X |
| Peak 8 | 29.329 | GI173367X |

The reconstituted ULTIVA® solution was diluted with mobile phase to provide an analytical solution of 0.5 mg/mL in pH 2.5 buffer. The stability of the analytical solution for ULTIVA® was performed according to the test method above. There were three impurities observed for the reconstituted ULTIVA® at initial time: carboxylic acid impurity (GR90291X) with a RRT of 0.8, the secondary amine (GR94219X), with a RRT of 0.7, formed via dealkylation at the piperidine nitrogen, and the impurity GR187657X with an RRT of 1.3. The amount of carboxylic acid impurity (GR90291X) formed was about 0.4% in 30 hours when stored at ambient room temperature. About 1.0 mL of the reconstituted solution was stored at 5° C., 25° C., 40° C., and 60° C. The stored solution was analyzed at time interval of 13 days using the test method. The stability results of reconstituted ULTIVA® in water are shown in TABLE 2. Review of the results in TABLE 2 shows that the purity of remifentanil at 40° C. was about 89% in 13 days. These results indicate that the stability of remifentanil in aqueous solution was less than 13 days and therefore not suitable for liquid formulation.

TABLE 2
Stability Date of Ultiva (Remifantil) reconstituted with water

| | | | % Area | | | |
|---|---|---|---|---|---|---|
| | | | | T = 13 | T = 13 | T = 13 |
| | | | | days at | days at | days at |
| No. | Name of Peak | RRT | T = 0 | 5° C. | 25° C. | 40° C. |
| 1 | Impurity | 0.6 | 0.11 | 0.09 | 0.10 | 0.08 |
| | GI114559X/ | | | | | |
| | GI128611X | | | | | |
| 3 | Impurity GR94219X | 0.7 | 0.15 | 0.14 | 0.15 | 0.24 |
| | (secondary amine) | | | | | |
| 4 | Impurity GR90291X | 0.8 | 0.43 | 0.75 | 2.22 | 9.81 |
| | (hydrolysis | | | | | |
| | degradant) | | | | | |
| 5 | RRT 0.9 Impurity | 0.9 | ND | ND | ND | ND |
| 6 | Remifentanil | 1.0 | 99.20 | 98.91 | 97.42 | 89.75 |
| 7 | RRT 1.1 Impurity | 1.1 | ND | ND | ND | ND |
| 8 | Impurity | 1.3 | 0.07 | 0.07 | 0.07 | 0.06 |
| | GR187657X | | | | | |
| 9 | Total Impurities | | 0.76 | 1.05 | 2.54 | 10.36 |

### Example 2

### Stability Study of Reconstituted ULTIVA® with PEG-400/PG at pH 4.5

Each of four (4) vials of ULTIVA® (Lot #61235 DD; Remifentanil HCl: 5 mg per vial; Lyophilized product; Manufacturer: Abbott; Exp. Date: 01/10, For R&D use only) was reconstituted with 5.0 mL of Polyethylene glycol-400/Propylene glycol (PG), 18:82, v/v (PEG-400/PG) at pH 2.5. The reconstituted solutions of ULTIVA® from four vials were pooled together to prepare a bulk solution of 1.0 mg/mL in PEG-400/PG. It is noted that 150 mL of PEG-400/PG was adjusted to pH 2.5 by adding 20 μL of 0.3 M hydrochloric acid. The pH of the reconstituted ULTIVA® solution in PEG-400/PG was 4.5. 1.0 mL of the reconstituted ULTIVA® product was transferred into glass vials and stored at various temperature conditions for the stability study of ULTIVA® in PEG-400/PG at pH 4.5. The following is the breakdown of the number of vials stored at each storage condition: six vials were stored at 5° C., six vials were stored at 25° C., and five vials were stored at 40° C. The vials were pulled from their respective storage conditions at various time intervals and analyzed.

The stability of the stored solution at 5° C., 25° C., 40° C., and 60° C. was analyzed at time intervals of 13 days and 20 days by the method set out in EXAMPLE 1. The stability results of reconstituted ULTIVA® in PEG-400/PG are shown in TABLE 3 below. Review of the results in TABLE 3 shows that the purity of remifentanil at 40° C. was about 89% in 13 days. An important observation was the formation of a new degradant impurity at RRT 0.9 with respect to remifentanil. The amount of RRT 0.9 impurity formed was 3.6% in 13 days at 40° C. storage conditions.

The peak at 0.9 RRT was characterized by LC-MS detection with ammonium acetate buffer as the mobile phase instead of ammonium phosphate buffer used for the analysis of stability samples. Review of the LC/MS data shows that the molecular weight of remifentanil peak was 376.8 which corresponds to the molecular weight of remifentanil. Likewise, the LC/MS data shows that the molecular weight of the unknown peak was 420.5 which corresponds to the molecular weight of the transesterified product of remifentanil with propylene glycol having the structures of IV and/or V set out above.

TABLE 3
Stability Data of Ultiva (Remifantil) reconstituted with PEG-400/PG at pH 4.5

| | | | % Area | | | | | | |
|---|---|---|---|---|---|---|---|---|---|
| | | | | 5° C. | | 25° C. | | 40° C. | 60° C. |
| | | | | T = 13 | T = 20 | T = 13 | T = 20 | T = 13 | T = 13 |
| No. | Name of Peak | RRT | T = 0 | days | days | days | days | days | days |
| 1 | Impurity GI114559X/ | 0.6 | ND | 0.08 | ND | 0.08 | ND | 0.20 | 2.42 |
| | GI128611X | | | | | | | | |
| 3 | Impurity GR94219X | 0.7 | 0.24 | 0.17 | 0.28 | 0.55 | 0.84 | 4.28 | 34.73 |
| | (secondary amine) | | | | | | | | |
| 4 | Impurity GR90291X | 0.8 | 0.42 | 0.43 | 0.43 | 0.44 | 0.48 | 0.55 | 1.44 |
| | (hydrolysis degradant) | | | | | | | | |
| 5 | RRT 0.9 Impurity | 0.9 | ND | 0.07 | 0.10 | 0.56 | 0.82 | 3.55 | 11.01 |
| 6 | Remifentanil | 1.0 | 98.98 | 98.99 | 99.05 | 98.07 | 97.37 | 88.87 | 40.84 |
| 7 | RRT 1.1 Impurity | 1.1 | ND | ND | ND | ND | 0.24 | ND | ND |
| 8 | Impurity GR187657X | 1.3 | ND | 0.20 | 0.08 | 0.19 | 0.21 | 1.68 | 4.30 |
| 9 | Total Impurities | | 1.0 | 0.95 | 0.94 | 1.88 | 2.64 | 10.26 | 53.90 |

### Example 3

### Stability Study of Reconstituted ULTIVA® with Glycerol

Each of the three (3) vials of ULTIVA® (Lot #61235 DD; Remifentanil HCl: 5 mg per vial; Lyophilized product; Manufacturer: Abbott; Exp. Date: 01/10, For R&D use only) was reconstituted with 3.0 mL of glycerol. The reconstituted solutions of ULTIVA® from three vials were pooled together to prepare a bulk solution in glycerine. About 300 μL of the reconstituted ULTIVA® solution was analyzed as time zero sample. The remaining solution was divided into 4 aliquots of about 2.0 mL and transferred into glass vials for storage, one vial each at 5° C., 25° C., 40° C. and 60° C. The vials were pulled from their respective storage conditions on Day 6 and analyzed.

The reconstituted ULTIVA® solution with glycerol formulation was diluted with mobile phase to provide an analytical solution of 0.5 mg/mL in pH 2.5 buffer. The stability of remifentanil in the analytical solution was analyzed by the method set out in EXAMPLE 1. There were five impurities observed for the reconstituted ULTIVA® in Glycerol at initial time. The impurities are carboxylic acid impurity (GR90291X) with a RRT of 0.8, and the secondary amine (GR94219X), with a RRT of 0.7, formed via dealkylation at the piperidine nitrogen. Additionally, GI128611X impurity at RRT 0.71, the impurity at GR187657X with RRT of 1.3 and impurity at RRT 1.1 were formed. The stability of the stored solution at 5° C., 25° C., 40° C., and 60° C. was analyzed at a time interval of 6 days. The stability results of reconstituted ULTIVA® in glycerol formulation are shown in Table 4. Review of the results in Table 4 shows that the purity of remifentanil at 60° C. was about 78% in 6 days. These results indicate a glycerol formulation does not provide adequate stability for remifentanil.

TABLE 4
Stability Data of Ultiva (Remifentanil) reconstituted with glycerol

| | | | % Assay¹/% Impurity² | | | | |
|---|---|---|---|---|---|---|---|
| | | | | T = 6 days | T = 6 days at | T = 6 days at | T = 6 days at |
| No. | Name of Peak | RRT | T = 0 | at 5° C. | 25° C. | 40° C. | 60° C. |
| 1 | Impurity | 0.71 | Not | Not | 0.07 | 1.21 | 11.55 |
| | GI114559X/ | | integrated | integrated | | | |
| | GI128611X | | | | | | |
| 3 | Impurity | 0.72 | Not | 0.14 | 0.16 | 0.41 | 8.63 |
| | GR94219X | | integrated | | | | |
| | (secondary | | | | | | |
| | amine) | | | | | | |
| 4 | Impurity | 0.8 | 0.47 | 0.44 | 0.39 | 0.54 | 0.48 |
| | GR90291X | | | | | | |
| | (hydrolysis | | | | | | |
| | degradant) | | | | | | |
| 5 | RRT 0.9 | 0.9 | ND | ND | ND | ND | ND |
| | Impurity | | | | | | |
| 6 | Remifentanil | 1.0 | 98.8 | 98.9 | 98.9 | 97.3 | 78.1 |
| 7 | RRT 1.1 | 1.1 | Not | Not | Not | 0.16 | 0.90 |
| | Impurity | | integrated | integrated | integrated | | |
| 8 | Impurity | 1.3 | 0.06 | 0.07 | 0.06 | 0.06 | 0.04 |
| | GR187657X | | | | | | |
| 9 | Total Impurities | | 1.20 | 0.69 | 1.28 | 2.38 | 21.6 |

¹Assay value corresponds to Remifentanil peak
²% Impurity value corresponds to peaks for impurities

### Example 4

### Formulation of Remifentanil in Glycerol

16.31 mg of remifentanil HCl was weighed and placed into a 10 mL volumetric flask and diluted to volume with glycerol. The solution was mixed well with sonication. The concentration of remifentanil HCl was 1.631 mg/mL. Three hundred μL of remifentanil solution was transferred to a HPLC vial and mixed with 700 μL of mobile phase to provide an analytical solution of 0.5 mg/mL in pH 2.5 buffer and analyzed according to the method set out in EXAMPLE 1. About 2 mL aliquots of remifentanil in glycerol formulation were transferred into glass vials and stored, one vial at each of the following storage conditions for stability testing: 5° C., 25° C., 40° C., and 60° C.

The stability of remifentanil in the analytical solution was then analyzed. There were five impurities observed for the remifentanil in glycerol formulation at initial time. Example chromatograms of remifentanil in glycerol and placebo for time zero and 6 day time interval showed various impurities: carboxylic acid impurity (GR90291X) with a RRT of 0.8, the secondary amine (GR94219X), with a RRT of 0.7, formed via dealkylation at the piperidine nitrogen, GI128611X impurity at RRT 0.71, the impurity at GR187657X with RRT of 1.3 and impurity at RRT 1.1 were formed. The stability of the stored solution at 5° C., 25° C., 40° C., and 60° C. was analyzed at time interval of 6 days by using client's method. The stability results of remifentanil in glycerol formulation are shown in TABLE 5 below and show that the purity of remifentanil at 60° C. was about 19% in 6 days. These results indicate remifentanil was not stable in glycerol formulation.

TABLE 5
Stability Data of Remifentanil in glycerol formulation

| | | | % Assay¹/% Impurity² | | | | |
|---|---|---|---|---|---|---|---|
| | | | | T = 6 days | T = 6 days at | T = 6 days | T = 6 days |
| No. | Name of Peak | RRT | T = 0 | at 5° C. | 25° C. | at 40° C. | at 60° C. |
| 1 | Impurity GI114559X/ | 0.71 | Not integrated | 0.30 | 2.28 | 12.36 | 22.88 |
| | GI128611X | | | | | | |
| 3 | Impurity GR94219X | 0.72 | Not integrated | 0.15 | Not | 4.69 | 54.17 |
| | (secondary amine) | | | | integrated | | |
| 4 | Impurity GR90291X | 0.8 | Not integrated | 0.05 | 0.06 | 0.13 | 0.50 |
| | (hydrolysis | | | | | | |
| | degradant) | | | | | | |
| 5 | RRT 0.9 Impurity | 0.9 | ND | ND | ND | ND | ND |
| 6 | Remifentanil | 1.0 | 98.9 | 98.7 | 97.2 | 81.8 | 19.1 |
| 7 | RRT 1.1 Impurity | 1.1 | Not integrated | Not | Not | 0.49 | 3.01 |
| | | | | integrated | integrated | | |
| 8 | Impurity | 1.3 | 0.09 | 0.10 | 0.09 | 0.08 | Not |
| | GR187657X | | | | | | integrated |
| 9 | Total Impurities | | Not integrated | 0.60 | 2.43 | 17.75 | 80.56 |

¹Assay value corresponds to Remifentanil peak
²% Impurity value corresponds to peaks for impurities

### Example 5

### Stability Study of Remifentanil in PEG-400/PG Constituted ULTIVA® with PEG-400/PG with and without Benzyl Alcohol

The stability of remifentanil in PEG-400 as well as the stability of remifentanil in propylene glycol (PG) were studied. Thirteen day testing of formulated remifentanil stored at 60° C. showed 88.3% remifentanil peak area for PEG-400 formulation and 68.8% remifentanil peak area for PG formulation of remifentanil. The stability of remifentanil in PEG-400/PG formulation with and without benzyl alcohol was analyzed by the test method set out in EXAMPLE 1. Remifentanil in PEG-400/PG formulations with and without benzyl alcohol were diluted with mobile phase to provide an analytical solution of 0.5 mg/mL in pH 2.5 buffer. There were two impurities observed for the remifentanil in PEG-400/PG formulations with and without benzyl alcohol at initial time—carboxylic acid impurity (GR90291X) with a RRT of 0.8, and the secondary amine (GR94219X), with a RRT of 0.7, formed via dealkylation at the piperidine nitrogen. The stability of the stored solution at 5° C., ambient laboratory conditions, 25° C., 40° C., and 60° C. was analyzed at time intervals of 6 days and 13 days. The stability results of remifentanil in PEG-400/PG formulation, with and without benzyl alcohol, are shown in Tables 6, 7, 8, 9 and 10 at 5° C., ambient laboratory conditions, 25° C., 40° C., and 60° C. conditions, respectively.

TABLE 6
STABILITY DATA OF REMIFENTANIL IN PEG-400/PPG
FORMULATION (18:82) AT 5° C.

| | | | % Assay¹/% Impurity² | | | % Assay¹/% Impurity² | | |
|---|---|---|---|---|---|---|---|---|
| | | | Formulation without Benzyl alcohol | | | Formulation with Benzyl alcohol | | |
| No. | Name of Peak | RRT | T = 0 | T = 6 Days | T = 13 Days | T = 0 | T = 6 Days | T = 13 Days |
| 1 | Impurity GI114559X | 0.6 | ND | ND | ND | ND | ND | ND |
| 2 | Impurity GI128611X | | | | 0.09 | | | ND |
| 3 | Impurity GR94219X | 0.7 | 0.07 | 0.15 | 0.30 | 0.07 | 0.16 | 0.32 |
| | (secondary amine) | | | | | | | |
| 4 | Impurity GR90291X | 0.8 | 0.18 | 0.06 | 0.06 | 0.18 | 0.05 | 0.06 |
| | (hydrolysis degradant) | | | | | | | |
| 5 | RRT 0.9 Impurity | 0.9 | ND | ND | 0.08 | ND | ND | Detected |
| 6 | Remifentanil Assay (% | 1.0 | 99.1 | 98.2 | 91.7 | 101.7 | 99.8 | 100.1 |
| | Theoretical) | | | | | | | |
| 7 | RRT 1.1 Impurity | 1.1 | ND | ND | ND | ND | ND | detected |
| 8 | Impurity GR187657X | 1.3 | ND | 0.14 | 0.17 | ND | masked by | masked by |
| | | | | | | | BzOH peak | BzOH peak |

¹Assay value corresponds to Remifentanil peak
²% Impurity value corresponds to peaks for impurities

TABLE 7
STABILITY DATA OF REMIFENTANIL IN PEG-400/PPG
FORMULATION (18:82) AT AMBIENT TEMPERATURE AND LIGHT
CONDITIONS

| | | | % Assay¹/% Impurity² | | | % Assay¹/% Impurity² | | |
|---|---|---|---|---|---|---|---|---|
| | | | Formulation without Benzyl alcohol | | | Formulation with Benzyl alcohol | | |
| No. | Name of Peak | RRT | T = 0 | T = 6 Days | T = 13 Days | T = 0 | T = 6 Days | T = 13 Days |
| 1 | Impurity GI114559X | 0.6 | ND | 0.07 | | ND | 0.04 | |
| 2 | Impurity GI128611X | | | | | | | |
| 3 | Impurity GR94219X | 0.7 | 0.07 | 0.32 | | 0.07 | 0.32 | |
| | (secondary amine) | | | | | | | |
| 4 | Impurity GR90291X | 0.8 | 0.18 | 0.05 | | 0.18 | 0.05 | |
| | (hydrolytic degradation) | | | | | | | |
| 5 | RRT 0.9 Impurity | 0.9 | ND | 0.21 | | ND | 0.23 | |
| 6 | Remifentanil Assay (% | 1.0 | 99.1 | 95.3 | | 101.7 | 97.6 | |
| | Theoretical) | | | | | | | |
| 7 | RRT 1.1 Impurity | 1.1 | ND | 0.07 | | ND | 0.06 | |
| 8 | Impurity GR187657X | 1.3 | 0.03 | 0.19 | | ND | masked by | |
| | | | | | | | BzOH peak | |

¹Assay value corresponds to Remifentanil peak
²% Impurity value corresponds to peaks for impurities

TABLE 8
STABILITY DATA OF REMIFENTANIL IN PEG-400/PPG
FORMULATION (18:82) AT 25° C.

| | | | % Assay¹/% Impurity² | | | % Assay¹/% Impurity² | | |
|---|---|---|---|---|---|---|---|---|
| | | | Formulation without Benzyl alcohol | | | Formulation with Benzyl alcohol | | |
| No. | Name of Peak | RRT | T = 0 | T = 6 Days | T = 13 Days | T = 0 | T = 6 Days | T = 13 Days |
| 1 | Impurity GI114559X | 0.6 | ND | 0.12 | ND | ND | 0.04 | detected |
| 2 | Impurity GI128611X | 0.6 | ND | ND | 0.11 | ND | ND | |
| 3 | Impurity GR94219X | 0.7 | 0.07 | 0.34 | 0.81 | 0.07 | 0.36 | detected |
| | (secondary amine) | | | | | | | |
| 4 | Impurity GR90291X | 0.8 | 0.18 | 0.05 | 0.06 | 0.18 | 0.05 | detected |
| | (hydrolytic degradation) | | | | | | | |
| 5 | RRT 0.9 Impurity | 0.9 | ND | 0.25 | 0.64 | ND | 0.29 | detected |
| 6 | Remifentanil Assay (% | 1.0 | 99.1 | 95.8 | 94.9 | 101.7 | 97.4 | 100.7 |
| | Theoretical) | | | | | | | |
| 7 | RRT 1.1 Impurity | 1.1 | ND | 0.07 | 0.21 | ND | 0.08 | detected |
| 8 | Impurity GR187657X | 1.3 | 0.03 | 0.85 | 0.38 | ND | masked by | masked by |
| | | | | | | | BzOH peak | BzOH peak |

¹This corresponds to Remifentanil peak
²This corresponds to Impurities peaks

TABLE 9
STABILITY DATA OF REMIFENTANIL IN PEG-400/PPG
FORMULATION (18:82) AT 40° C.

| | | | % Assay¹/% Impurity² | | | % Assay¹/% Impurity² | | |
|---|---|---|---|---|---|---|---|---|
| | | | Formulation without Benzyl alcohol | | | Formulation with Benzyl alcohol | | |
| No. | Name of Peak | RRT | T = 0 | T = 6 Days | T = 13 Days | T = 0 | T = 6 Days | T = 13 Days |
| 1 | Impurity GI114559X | 0.6 | ND | ND | ND | ND | ND | |
| 2 | Impurity GI128611X | 0.6 | ND | ND | 0.11 | ND | ND | |
| 3 | Impurity GR94219X | 0.7 | 0.07 | 2.24 | 5.61 | 0.07 | 2.43 | detected |
| | (secondary amine) | | | | | | | |
| 4 | Impurity GR90291X | 0.8 | 0.18 | 0.11 | 0.23 | 0.18 | 0.07 | detected |
| | (hydrolytic | | | | | | | |
| | degradation) | | | | | | | |
| 5 | RRT 0.9 Impurity | 0.9 | ND | 1.65 | 4.14 | ND | 1.65 | detected |
| 6. | Remifentanil Assay | 1.0 | 99.1 | 90.3 | 84.1 | 101.7 | 93.3 | 87.5 |
| | (% Theoretical) | | | | | | | |
| 7 | RRT 1.1 Impurity | 1.1 | ND | 0.83 | 2.04 | ND | 0.83 | detected |
| 8 | Impurity GR187657X | 1.3 | 0.03 | 0.38 | 0.73 | ND | masked by | masked by |
| | | | | | | | BzOH peak | BzOH |

¹This corresponds to Remifentanil peak
²This corresponds to Impurities peaks

TABLE 10
STABILITY DATA OF REMIFENTANIL IN PEG-400/PPG
FORMULATION (18:82) AT 60° C.

| | | | % Assay¹/% Impurity² | | | % Assay¹/% Impurity² | | |
|---|---|---|---|---|---|---|---|---|
| | | | Formulation without Benzyl alcohol | | | Formulation with Benzyl alcohol | | |
| No. | Name of Peak | RRT | T = 0 | T = 6 Days | T = 13 Days | T = 0 | T = 6 Days | T = 13 Days |
| 1 | Impurity GI114559X/ | 0.6 | ND | ND | 0.11 | ND | ND | detected |
| | GI128611X | | | | | | | |
| 2 | Impurity GR94219X | 0.7 | 0.07 | 13.29 | 22.87 | 0.07 | 13.13 | 19.63 |
| | (secondary amine) | | | | | | | |
| 3 | Impurity GR90291X | 0.8 | 0.18 | 0.43 | 1.04 | 0.18 | 0.24 | 0.70 |
| | (hydrolytic degradation) | | | | | | | |
| 4 | RRT 0.9 Impurity | 0.9 | ND | 6.95 | detected | ND | 6.95 | detected |
| 5. | Remifentanil Assay (% | 1.0 | 99.1 | 70.8 | 53.6 | 101.7 | 76.3 | 59.3 |
| | Theoretical) | | | | | | | |
| 6 | RRT 1.1 Impurity | 1.1 | ND | 4.94 | detected | ND | 4.94 | detected |
| 7 | Impurity GR187657X | 1.3 | 0.03 | 0.71 | | ND | masked by | masked by |
| | | | | | | | BzOH peak | BzOH peak |

¹This corresponds to Remifentanil peak
²This corresponds to Impurities peaks

Review of the results in Table 10 shows that the purity of remifentanil at 60° C. was about 54% in 13 days for PEG-400/PG formulation without benzyl alcohol and the purity of remifentanil at 60° C. was about 59% in 13 days for PEG-400/PG formulation with benzyl alcohol. Both PEG-400/PG formulations with and without benzyl alcohol showed the presence of an impurity at RRT 0.9 with respect to remifentanil peak. PEG-400/PG formulation without benzyl alcohol also showed the presence of impurity GR187657X. The presence of impurity GR187657X in PEG-400/PG formulation with benzyl alcohol could not be determined due to the presence of benzyl alcohol peak at the same retention time for GR187657X. The interference of benzyl alcohol peak was determined from the analysis of the corresponding placebo sample.

### Example 6

### Stability Study of Remifentanil in PEG-400/PG Formulation at pH 2.5 with and without Glycine (Non-Aqueous Formulation)

The stability of remifentanil in PEG-400/PG formulation at pH 2.5 with and without glycine was analyzed by using the test method set out in EXAMPLE 1. Remifentanil in PEG-400/PG formulation at pH 2.5 with and without glycine was diluted with mobile phase to provide an analytical solution of 0.5 mg/mL in pH 2.5 buffer. There were three impurities observed by HPLC for the remifentanil in PEG-400/PG formulation at pH 2.5 with and without glycine at initial time. The impurities are carboxylic acid impurity (GR90291X) with a RRT of 0.8, the secondary amine (GR94219X), with a RRT of 0.7, and the impurity GR187657X with a RRT of 1.3. The stability of the stored solution at 5° C., 25° C., 40° C., and 60° C. was analyzed at time interval of 5 days and 28 days. The stability results of remifentanil in PEG-400/PG formulation at pH 2.5, with and without glycine, are shown in TABLES 11, 12, 13 and 14 at 5° C., 25° C., 40° C., and 60° C. conditions, respectively.

TABLE 11
Stability Data of Remifentanil in PEG-400/PPG Formulation of pH 2.5 at 5° C.

| | | | % Assay¹/% Impurity² | | | % Assay¹/% Impurity² | | |
|---|---|---|---|---|---|---|---|---|
| | | | Formulation without Glycine | | | Formulation with Glycine | | |
| No. | Name of Peak | RRT | T = 0 | T = 5 Days | T = 28 Days³ | T = 0 | T = 5 Days | T = 28 Days |
| 1 | Impurity GI114559X | 0.6 | ND | ND | ND | ND | ND | |
| 2 | Impurity GI128611X | | | | | | | |
| 3 | Impurity GR94219X | 0.7 | 0.114 | 0.12 | 0.25 | 0.143 | 0.15 | |
| | (secondary amine) | | | | | | | |
| 4 | Impurity GR90291X | 0.8 | 0.043 | 0.05 | 0.05 | 0.055 | 0.05 | |
| | (hydrolysis degradant) | | | | | | | |
| 5 | RRT 0.9 Impurity | 0.9 | ND | ND | 0.03 | ND | ND | |
| | Remifentanil (% Area) | | | | 99.2 | | | |
| 6 | Remifentanil Assay (% | 1.0 | 90.9 | 92.7 | — | 99.6 | 101.2 | |
| | Theoretical) | | | | | | | |
| 7 | RRT 1.1 Impurity | 1.1 | ND | ND | ND | ND | ND | |
| 8 | Impurity GR187657X | 1.3 | 0.09 | 0.14 | 0.11 | 0.10 | 0.15 | |

¹Assay value corresponds to Remifentanil peak
²% Impurity value corresponds to peaks for impurities
³% Area/Area

TABLE 12
Stability Data of Remifentanil in PEG-400/PPG Formulation of pH 2.5 at 25° C.

| | | | % Assay¹/% Impurity² | | | % Assay¹/% Impurity² | | |
|---|---|---|---|---|---|---|---|---|
| | | | Formulation without glycine | | | Formulation with glycine | | |
| | | | | T = 5 | T = 28 | | T = 5 | T = 28 |
| No. | Name of Peak | RRT | T = 0 | Days | Days | T = 0 | Days | Days |
| 1 | Impurity GI114559X | 0.6 | ND | ND | | ND | ND | |
| 2 | Impurity GI128611X | 0.6 | | | | | | |
| 3 | Impurity GR94219X | 0.7 | 0.114 | 0.13 | 0.26 | 0.143 | 0.16 | |
| | (secondary amine) | | | | | | | |
| 4 | Impurity GR90291X | 0.8 | 0.043 | 0.06 | 0.06 | 0.055 | 0.06 | |
| | (hydrolytic degradation) | | | | | | | |
| 5 | RRT 0.9 Impurity | 0.9 | ND | ND | 0.04 | ND | 0.06 | |
| | Remifentanil (% Area) | | | | 99.19 | | | |
| 6 | Remifentanil Assay | 1.0 | 90.9 | 99.0 | — | 99.6 | 100.4 | |
| | (% Theoretical) | | | | | | | |
| 7 | RRT 1.1 Impurity | 1.1 | ND | ND | ND | ND | ND | |
| 8 | Impurity GR187657X | 1.3 | 0.09 | 0.12 | 0.11 | 0.10 | 0.14 | |

¹This corresponds to Remifentanil peak
²This corresponds to Impurities peaks

TABLE 13
Stability Data of Remifentanil in PEG-400/PPG Formulation of pH 2.5 at 40° C.

| | | | % Assay¹/% Impurity² | | | % Assay¹/% Impurity² | | |
|---|---|---|---|---|---|---|---|---|
| | | | Formulation without glycine | | | Formulation with glycine | | |
| | | | | T = 5 | T = 28 | | T = 5 | T = 28 |
| No. | Name of Peak | RRT | T = 0 | Days | Days | T = 0 | Days | Days |
| 1. | Impurity GI114559X | 0.6 | ND | ND | — | ND | ND | |
| 2. | Impurity GI128611X | 0.6 | | | | | | |
| 3. | Impurity GR94219X | 0.7 | 0.11 | 0.15 | 0.35 | 0.14 | 0.22 | |
| | (secondary amine) | | | | | | | |
| 4. | Impurity GR90291X | 0.8 | 0.043 | 0.07 | 0.16 | 0.055 | 3.03 | |
| | (hydrolytic degradation) | | | | | | | |
| 5. | RRT 0.9 Impurity | 0.9 | ND | 0.04 | 0.28 | ND | 0.31 | |
| | Remifentanil (% Area) | | | | 98.82 | | | |
| 6. | Remifentanil Assay | 1.0 | 90.9 | 96.4 | — | 99.6 | 97.6 | |
| | (% Theoretical) | | | | | | | |
| 7. | RRT 1.1 Impurity | 1.1 | ND | ND | ND | ND | ND | |
| 8. | Impurity GR187657X | 1.3 | 0.09 | 0.11 | 0.11 | 0.10 | 0.16 | |

¹This corresponds to Remifentanil peak
²This corresponds to Impurities peaks

TABLE 14
Stability Data of Remifentanil in PEG-400/PPG Formulation of pH 2.5 at 60° C.

| | | | % Assay¹/% Impurity² | | | % Assay¹/% Impurity² | | |
|---|---|---|---|---|---|---|---|---|
| | | | Formulation without glycine | | | Formulation with glycine | | |
| | | | | T = 5 | T = 28 | | T = 5 | T = 28 |
| No. | Name of Peak | RRT | T = 0 | Days | Days | T = 0 | Days | Days |
| 1 | Impurity GI114559X/ | 0.6 | ND | ND | ND | ND | 0.05 | |
| | GI128611X | | | | | | | |
| 2 | Impurity GR94219X | 0.7 | 0.114 | 0.24 | 4.68 | 0.143 | 1.26 | |
| | (secondary amine) | | | | | | | |
| 3 | Impurity GR90291X | 0.8 | 0.043 | 0.17 | 0.87 | 0.055 | 13.86 | |
| | (hydrolytic degradation) | | | | | | | |
| 4 | RRT 0.9 Impurity | 0.9 | ND | 0.28 | 5.52 | ND | 1.72 | |
| 5 | Remifentanil (% Area) | | | | 79.62 | | | |
| | Remifentanil Assay | 1.0 | 90.9 | 91.7 | — | 99.6 | 84.7 | |
| | (% Theoretical) | | | | | | | |
| 6 | RRT 1.1 Impurity | 1.1 | ND | ND | ND | ND | ND | |
| 7 | Impurity GR187657X | 1.3 | 0.09 | 0.10 | ND | 0.10 | 0.10 | |

¹This corresponds to Remifentanil peak
²This corresponds to Impurities peaks

Review of the results in Table 14 shows that the amount of degradant (GR90291X) for PEG-400/PG formulation at pH 2.5 with glycine was about 14% for 5 days time point at 60° C., and the amount of degradant (GR90291X) for PEG-400/PG formulation at pH 2.5 without glycine was about 0.2% in 5 days time at 60° C.

### Example 7

### Stability Study of Remifentanil in PEG-400/PG Formulation at pH 2.0, pH 2.5 and pH 3.0 (Non-Aqueous Formulation)

The stability of remifentanil in PEG-400/PG formulation at pH 2.0, 2.5, and 3.0 was analyzed by using the EXAMPLE 1 test method. Remifentanil in PEG-400/PG formulation at pH 2.0, 2.5, and 3.0 was diluted with mobile phase to provide an analytical solution of 0.5 mg/mL in pH 2.5 buffer. Three impurities were observed for all three formulations of remifentanil in PEG-400/PG formulation at initial time. High Performance Liquid Chromatography (HPLC) chromatograms of remifentanil in PEG-400/PG formulation at pH 2.0, pH 2.5, and pH 3.0 and its placebos showed the following impurities: carboxylic acid impurity (GR90291X) with a RRT of 0.8, the secondary amine (GR94219X), with a RRT of 0.7, formed via dealkylation at the piperidine nitrogen, and the impurity GR187657X with a RRT of 1.3. The stability of the stored solution at 5° C., 25° C., 40° C., and 60° C. was analyzed at time interval of 1 week, 2 weeks, 4 weeks, 8 weeks, 12 weeks, 15 weeks, and 7 months. Analysis was performed using the EXAMPLE 1 test method for time zero (initial time point), and time intervals of 1 week, 2 weeks, 4 weeks, 8 weeks, and 12 weeks. Additional development work was performed for a method used in the analysis of 15 week and 7 month time intervals. Chromatograms of the analysis of 7 month samples were obtained and the stability results of remifentanil in PEG-400/PG formulation at pH 2.0 are shown in TABLES 15, 16, 17, and 18 for 5° C., 25° C., 40° C., and 60° C. conditions, respectively. The stability results of remifentanil in PEG-400/PG formulation at pH 2.5 are shown in TABLES 19, 20, 21 and 22 for 5° C., 25° C., 40° C., and 60° C. conditions, respectively.

The stability results of remifentanil in PEG-400/PG formulation at pH 3.0 are shown in TABLES 23, 24, 25 and 26 for 5° C., 25° C., 40° C., and 60° C. conditions, respectively.

TABLE 15
Stability Data of Remifentanil in PEG-400/PG Formulation at pH 2.0 at 5° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks³ | weeks³ | weeks | weeks | months |
| 1 | Impurity GI114559X/ | 0.6 | ND | ND | ND | ND | ND | ND | ND | ND |
| | GI128611X | | | | | | | | | |
| 3 | Impurity GR94219X | 0.7 | 0.10 | 0.11 | 0.19 | 0.15 | 0.17 | 0.15 | 0.11 | 0.14 |
| | (secondary amine) | | | | | | | | | |
| 4 | Impurity GR90291X | 0.8 | 0.06 | 0.06 | 0.05 | 0.06 | 0.08 | 0.07 | 0.11 | 0.09 |
| | (hydrolysis degradant) | | | | | | | | | |
| 5 | RRT 0.9 Impurity | 0.9 | ND | ND | ND | ND | 0.5 | 0.04 | ND | ND |
| 6 | Remifentanil Assay | 1.0 | 102.1 | 104.2 | 101.0 | 99.3 | 97.2 | 95.4 | 97.2 | 101.5 |
| | (% Theoretical) | | | | | | | | | |
| 7 | RRT 1.1 Impurity | 1.1 | ND | ND | ND | ND | ND | ND | ND | ND |
| 8 | Impurity GR187657X | 1.3 | 0.12 | 0.10 | 0.07 | 0.15 | 0.09 | 0.08 | ND | 0.11 |
| 9 | Total Impurities | | 0.28 | 0.27 | 0.31 | 0.36 | 0.83 | 0.34 | 0.22 | 0.34 |

¹Assay value corresponds to Remifentanil peak
²% Impurity value corresponds to peaks for impurities
³Partial vial of product was used for testing

TABLE 16
Stability Data of Remifentanil in PEG-400/PG Formulation at pH 2.0 at 25° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks | weeks | weeks | weeks | months |
| 1 | Impurity GI114559X/ | 0.6 | ND | ND | ND | ND | ND | ND | ND | ND |
| | GI128611X | | | | | | | | | |
| 3 | Impurity GR94219X | 0.7 | 0.10 | 0.11 | 0.19 | 0.15 | 0.17 | 0.15 | 0.12 | 0.14 |
| | (secondary amine) | | | | | | | | | |
| 4 | Impurity GR90291X | 0.8 | 0.06 | 0.06 | 0.06 | 0.07 | 0.07 | 0.09 | 0.14 | 0.13 |
| | (hydrolysis degradant) | | | | | | | | | |
| 5 | RRT 0.9 Impurity | 0.9 | ND | ND | 0.03 | 0.07 | 0.60 | 0.16 | ND | ND |
| 6 | Remifentanil Assay | 1.0 | 102.1 | 101.4 | 101.3 | 99.3 | 97.8 | 94.0 | 95.53 | 98.0 |
| | (% Theoretical) | | | | | | | | | |
| 7 | RRT 1.1 Impurity | 1.1 | ND | ND | ND | ND | ND | ND | 0.06 | 0.41 |
| 8 | Impurity GR187657X | 1.3 | 0.12 | 0.10 | 0.09 | 0.16 | 0.09 | 0.07 | 0.09 | 0.09 |
| 9 | Total Impurities | | 0.28 | 0.26 | 0.36 | 0.45 | 0.95 | 0.48 | 0.41 | 0.99 |

¹Assay value corresponds to Remifentanil peak
²% Impurity value corresponds to peaks for impurities

TABLE 17
Stability Data of Remifentanil in PEG-400/PG Formulation at pH 2.0 at 40° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks³ | weeks³ | weeks | weeks | months |
| 1 | Impurity GI114559X/ | 0.6 | ND | ND | ND | ND | ND | ND | 0.07 | 0.24 |
| | GI128611X | | | | | | | | | |
| 3 | Impurity GR94219X | 0.7 | 0.10 | 0.11 | 0.20 | 0.16 | 0.23 | 0.23 | 0.26 | 0.52 |
| | (secondary amine) | | | | | | | | | |
| 4 | Impurity GR90291X | 0.8 | 0.06 | 0.06 | 0.08 | 0.12 | 0.17 | 0.20 | 0.23 | 0.32 |
| | (hydrolysis degradant) | | | | | | | | | |
| 5 | RRT 0.9 Impurity | 0.9 | ND | ND | 0.16 | 0.31 | 0.92 | 0.76 | ND | ND |
| 6 | Remifentanil Assay | 1.0 | 102.1 | 100.4 | 100.9 | 97.1 | 96.3 | 92.8 | 96.15 | 96.6 |
| | (% Theoretical) | | | | | | | | | |
| 7 | RRT 1.1 Impurity | 1.1 | ND | ND | ND | ND | ND | 0.07 | 0.42 | 1.10 |
| 8 | Impurity GR187657X | 1.3 | 0.12 | 0.09 | 0.08 | 0.15 | 0.08 | 0.06 | 0.04 | 0.08 |
| 9 | Total Impurities | | 0.28 | 0.28 | 0.53 | 0.74 | 1.40 | 1.69 | 1.03 | 2.61 |

¹Assay value corresponds to Remifentanil peak
²% Impurity value corresponds to peaks for impurities
³Partial vial of product was used for testing

TABLE 18
Stability Data of Remifentanil in PEG-400/PG Formulation at pH 2.0 at 60° C.

| | | | % Assay¹/% Impurity² | | | | |
|---|---|---|---|---|---|---|---|
| No. | Name of Peak | RRT | T = 0 | T = 1 week | T = 2 weeks | T = 4 weeks³ | T = 8 weeks³ |
| 1 | Impurity GI114559X/ | 0.6 | ND | ND | ND | ND | ND |
| | GI128611X | | | | | | |
| 3 | Impurity GR94219X | 0.7 | 0.10 | 0.25 | 0.38 | 1.41 | 4.37 |
| | (secondary amine) | | | | | | |
| 4 | Impurity GR90291X | 0.8 | 0.06 | 0.14 | 0.17 | 0.54 | 1.28 |
| | (hydrolysis degradant) | | | | | | |
| 5 | RRT 0.9 Impurity | 0.9 | ND | 0.28 | 0.76 | 2.14 | 67.8 |
| 6 | Remifentanil Assay | 1.0 | 102.1 | 100.4 | 100.0 | 88.5 | 67.8 |
| | (% Theoretical) | | | | | | |
| 7 | RRT 1.1 Impurity | 1.1 | ND | ND | ND | ND | ND |
| 8 | Impurity GR187657X | 1.3 | 0.12 | 0.08 | 0.07 | 0.11 | 0.02 |
| 9 | Total Impurities | | 0.28 | 0.78 | 1.70 | 6.66 | 24.7 |

¹Assay value corresponds to Remifentanil peak
²% Impurity value corresponds to peaks for impurities
³Partial vial of product was used for testing

TABLE 19
Stability Data of Remifentanil in PEG-400/PG Formulation at pH 2.5 at 5° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks³ | weeks³ | weeks | weeks | months |
| 1 | Impurity GI114559X/ | 0.6 | ND | ND | ND | ND | ND | ND | ND | ND |
| | GI128611X | | | | | | | | | |
| 3 | Impurity GR94219X | 0.7 | 0.10 | 0.10 | 0.18 | 0.16 | 0.18 | 0.13 | 0.11 | 0.14 |
| | (secondary amine) | | | | | | | | | |
| 4 | Impurity GR90291X | 0.8 | 0.06 | 0.06 | 0.06 | 0.06 | 0.07 | 0.07 | 0.11 | 0.06 |
| | (hydrolysis degradant) | | | | | | | | | |
| 5 | RRT 0.9 Impurity | 0.9 | ND | ND | ND | ND | 0.25 | 0.01 | ND | ND |
| 6 | Remifentanil Assay | 1.0 | 103.0 | 103.6 | 102.1 | 100.0 | 98.6 | 94.7 | 97.10 | 99.5 |
| | (% Theoretical) | | | | | | | | | |
| 7 | RRT 1.1 Impurity | 1.1 | ND | ND | ND | ND | ND | ND | ND | 0.26 |
| 8 | Impurity GR187657X | 1.3 | 0.12 | 0.10 | 0.10 | 0.11 | 0.10 | 0.08 | ND | 0.10 |
| 9 | Total Impurities | | 0.28 | 0.27 | 0.35 | 0.28 | 0.59 | 0.30 | 0.22 | 0.79 |

¹Assay value corresponds to Remifentanil peak
²% Impurity value corresponds to peaks for impurities
³Partial vial of product was used for testing

TABLE 20
Stability Data of Remifentanil in PEG-400/PG Formulation at pH 2.50 at 25° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks | weeks | weeks | weeks | months |
| 1 | Impurity GI114559X/ | 0.6 | ND | ND | ND | ND | ND | ND | ND | 0.03 |
| | GI128611X | | | | | | | | | |
| 3 | Impurity GR94219X | 0.7 | 0.10 | 0.10 | 0.18 | 0.17 | 0.18 | 0.16 | 0.10 | 0.18 |
| | (secondary amine) | | | | | | | | | |
| 4 | Impurity GR90291X | 0.8 | 0.06 | 0.06 | 0.06 | 0.06 | 0.07 | 0.06 | 0.10 | 0.07 |
| | (hydrolysis degradant) | | | | | | | | | |
| 5 | RRT 0.9 Impurity | 0.9 | ND | ND | ND | 0.03 | 0.23 | 0.07 | ND | ND |
| 6 | Remifentanil Assay | 1.0 | 103.0 | 102.0 | 102.2 | 100.6 | 97.8 | 94.3 | 97.2 | 97.9 |
| | (% Theoretical) | | | | | | | | | |
| 7 | RRT 1.1 Impurity | 1.1 | ND | ND | ND | 0.11 | ND | ND | ND | 0.40 |
| 8 | Impurity GR187657X | 1.3 | 0.12 | 0.10 | 0.10 | ND | 0.11 | 0.08 | 0.09 | 0.09 |
| 9 | Total Impurities | | 0.28 | 0.26 | 0.34 | 0.28 | 0.58 | 0.38 | 0.32 | 1.05 |

¹Assay value corresponds to Remifentanil peak
²% Impurity value corresponds to peaks for impurities

TABLE 21
Stability Data of Remifentanil in PEG-400/PG Formulation at pH 2.50 at 40° C.

| | | | % Assay¹/% Impurity² | | | | | | |
|---|---|---|---|---|---|---|---|---|---|
| | | | | T = 1 | T = 2 | T = 4 | T = 8 | T = 12 | T = 15 |
| No. | Name of Peak | RRT | T = 0 | week | weeks | weeks | weeks | weeks | weeks |
| 1 | Impurity GI114559X/ | 0.5 | ND | ND | ND | ND | ND | ND | 0.34 |
| | GI128611X | | | | | | | | |
| 3 | Impurity GR94219X | 0.7 | 0.10 | 0.13 | 0.23 | 0.29 | 0.50 | 0.60 | 0.90 |
| | (secondary amine) | | | | | | | | |
| 4 | Impurity GR90291X | 0.8 | 0.06 | 0.07 | 0.07 | 0.07 | 0.10 | 0.10 | 0.14 |
| | (hydrolysis degradant) | | | | | | | | |
| 5 | RRT 0.9 Impurity | 0.9 | ND | 0.02 | 0.12 | 0.25 | 0.75 | 0.69 | ND |
| 6 | Remifentanil Assay | 1.0 | 103.0 | 102.6 | 101.8 | 99.8 | 97.8 | 92.5 | 95.8 |
| | (% Theoretical) | | | | | | | | |
| 7 | RRT 1.1 Impurity | 1.1 | ND | ND | ND | ND | 0.42 | 0.26 | 0.53 |
| 8 | Impurity GR187657X | 1.3 | 0.12 | 0.09 | 0.10 | 0.08 | 0.11 | 0.07 | 0.13 |
| 9 | Total Impurities | | 0.28 | 0.32 | 0.52 | 0.68 | 1.88 | 1.83 | 2.06 |

¹Assay value corresponds to Remifentanil peak
²% Impurity value corresponds to peaks for impurities

TABLE 22
Stability Data of Remifentanil in PEG-400/PG Formulation at pH 2.50 at 60° C.

| | | | % Assay¹/% Impurity² | | | | |
|---|---|---|---|---|---|---|---|
| No. | Name of Peak | RRT | T = 0 | T = 1 week | T = 2 weeks | T = 4 weeks³ | T = 8 weeks³ |
| 1 | Impurity GI114559X/ | 0.6 | ND | ND | 0.07 | ND | ND |
| | GI128611X | | | | | | |
| 3 | Impurity GR94219X | 0.7 | 0.10 | 0.71 | 1.43 | 1.53 | 4.83 |
| | (secondary amine) | | | | | | |
| 4 | Impurity GR90291X | 0.8 | 0.06 | 0.09 | 0.22 | 1.08 | 1.54 |
| | (hydrolysis degradant) | | | | | | |
| 5 | RRT 0.9 Impurity | 0.9 | ND | 0.48 | 1.19 | 4.08 | 8.34 |
| 6 | Remifentanil Assay | 1.0 | 103.0 | 99.8 | 101.8 | 77.8 | 55.4 |
| | (% Theoretical) | | | | | | |
| 7 | RRT 1.1 Impurity | 1.1 | ND | 0.24 | ND | 0.04 | 0.12 |
| 8 | Impurity GR187657X | 1.3 | 0.12 | 0.06 | 0.06 | 0.08 | 0.03 |
| 9 | Total Impurities | | 0.28 | 1.74 | 3.66 | 16.56 | 37.26 |

¹Assay value corresponds to Remifentanil peak
²% Impurity value corresponds to peaks for impurities
³Partial vial of product was used for testing.

TABLE 23
Stability Data of Remifentanil in PEG-400/PG Formulation at pH 3.0 at 5° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks_{³} | weeks_{³} | weeks | weeks | months |
| 1 | Impurity GI114559X/ | 0.6 | ND | ND | ND | ND | ND | ND | ND | ND |
| | GI128611X | | | | | | | | | |
| 3 | Impurity GR94219X | 0.7 | 0.10 | 0.10 | 0.11 | 0.15 | 0.17 | 0.13 | 0.11 | 0.15 |
| | (secondary amine) | | | | | | | | | |
| 4 | Impurity GR90291X | 0.8 | 0.06 | 0.06 | 0.06 | 0.06 | 0.06 | 0.06 | 0.08 | 0.06 |
| | (hydrolysis degradant) | | | | | | | | | |
| 5 | RRT 0.9 Impurity | 0.9 | ND | ND | ND | ND | 0.50 | 0.02 | ND | ND |
| 6 | Remifentanil Assay | 1.0 | 106.1 | 100.5 | 98.6 | 95.8 | 95.3 | 91.1 | 94.2 | 93.6 |
| | (% Theoretical) | | | | | | | | | |
| 7 | RRT 1.1 Impurity | 1.1 | ND | ND | ND | ND | ND | ND | ND | 0.06 |
| 8 | Impurity GR187657X | 1.3 | 0.10 | 0.10 | 0.10 | 0.16 | ND | 0.07 | ND | 0.10 |
| 9 | Total Impurities | | 0.26 | 0.26 | 0.27 | 0.37 | 0.83 | 0.28 | 0.19 | 0.37 |

¹Assay value corresponds to Remifentanil peak
²% Impurity value corresponds to peaks for impurities
3Partial vial of product was used for testing

TABLE 24
Stability Data of Remifentanil in PEG-400/PG Formulation at pH 3.0 at 25° C.

| | | | % Assay¹/% Impurity² | | | | | | |
|---|---|---|---|---|---|---|---|---|---|
| | | | | T = 1 | T = 2 | T = 4 | T = 8 | T = 12 | T = 15 |
| No. | Name of Peak | RRT | T = 0 | week | weeks | weeks | weeks | weeks | weeks³ |
| 1 | Impurity GI114559X/ | 0.6 | ND | ND | ND | ND | ND | ND | 0.03 |
| | GI128611X | | | | | | | | |
| 3 | Impurity GR94219X | 0.7 | 0.10 | 0.11 | 0.13 | 0.19 | 0.22 | 0.21 | 0.22 |
| | (secondary amine) | | | | | | | | |
| 4 | Impurity GR90291X | 0.8 | 0.06 | 0.06 | 0.07 | 0.07 | 0.06 | 0.07 | 0.08 |
| | (hydrolysis degradant) | | | | | | | | |
| 5 | RRT 0.9 Impurity | 0.9 | ND | ND | 0.02 | 0.05 | 0.62 | 0.14 | ND |
| 6 | Remifentanil Assay | 1.0 | 106.1 | 102.2 | 105.8 | 103.3 | 95.7 | 93.3 | 97.8 |
| | (% Theoretical) | | | | | | | | |
| 7 | RRT 1.1 Impurity | 1.1 | ND | ND | ND | ND | ND | 0.06 | 0.08 |
| 8 | Impurity GR187657X | 1.3 | 0.10 | 0.08 | 0.10 | 0.16 | 0.11 | 0.07 | 0.07 |
| 9 | Total Impurities | | 0.26 | 0.24 | 0.32 | 0.37 | 1.02 | 0.55 | 0.48 |

¹Assay value corresponds to Remifentanil peak
²% Impurity value corresponds to peaks for impurities
³Stability Testing stopped after 15 weeks

TABLE 25
Stability Data of Remifentanil in PEG-400/PG Formulation at pH 3.0 at 40° C.

| | | | % Assay¹/% Impurity² | | | | | | | |
|---|---|---|---|---|---|---|---|---|---|---|
| | | | | T = 1 | T = 2 | T = 4 | T = 8 | T = 12 | T = 15 | T = 7 |
| No. | Name of Peak | RRT | T = 0 | week | weeks | weeks³ | weeks³ | weeks | weeks | months |
| 1 | Impurity GI114559X/ | 0.5 | ND | ND | 0.02 | ND | ND | ND | 0.60 | ND |
| | GI128611X | | | | | | | | | |
| 3 | Impurity GR94219X | 0.7 | 0.10 | 0.20 | 0.41 | 0.76 | 1.45 | 1.46 | 1.83 | 2.03 |
| | (secondary amine) | | | | | | | | | |
| 4 | Impurity GR90291X | 0.8 | 0.06 | 0.06 | 0.07 | 0.09 | 0.12 | 0.13 | 0.22 | 0.54 |
| | (hydrolysis degradant) | | | | | | | | | |
| 5 | RRT 0.9 Impurity | 0.9 | ND | ND | 0.30 | 0.64 | 1.83 | 1.37 | ND | ND |
| 6 | Remifentanil Assay | 1.0 | 106.1 | 104.0 | 103.4 | 100.8 | 97.3 | 98.0 | 94.8 | 93.8 |
| | (% Theoretical) | | | | | | | | | |
| 7 | RRT 1.1 Impurity | 1.1 | ND | ND | 0.03 | ND | 0.82 | 0.64 | 1.02 | 2.78 |
| 8 | Impurity GR187657X | 1.3 | 0.10 | 0.07 | 0.09 | 0.14 | 0.16 | 0.07 | 0.38 | 0.08 |
| 9 | Total Impurities | | 0.26 | 0.34 | 0.93 | 1.67 | 4.50 | 4.07 | 4.06 | 6.82 |

¹Assay value corresponds to Remifentanil peak
²% Impurity value corresponds to peaks for impurities
³Partial vial of product was used for testing

TABLE 26
Stability Data of Remifentanil in PEG-400/PG Formulation at pH 3.0 at 60° C.

| | | | % Assay¹/% Impurity² | | | | |
|---|---|---|---|---|---|---|---|
| | | | | T = 1 | T = 2 | T = 4 | T = 8 |
| No. | Name of Peak | RRT | T = 0 | week | weeks | weeks³ | weeks³ |
| 1 | Impurity GI114559X/ | 0.6 | ND | ND | 0.11 | ND | ND |
| | GI128611X | | | | | | |
| 3 | Impurity GR94219X | 0.7 | 0.10 | 1.38 | 2.26 | 1.50 | 3.85 |
| | (secondary amine) | | | | | | |
| 4 | Impurity GR90291X | 0.8 | 0.06 | 0.11 | 0.24 | 0.88 | 1.54 |
| | (hydrolysis degradant) | | | | | | |
| 5 | RRT 0.9 Impurity | 0.9 | ND | 0.68 | 1.67 | 3.08 | 6.88 |
| 6 | Remifentanil Assay | 1.0 | 106.1 | 103.0 | 100.4 | 86.3 | 64.6 |
| | (% Theoretical) | | | | | | |
| 7 | RRT 1.1 Impurity | 1.1 | ND | 0.61 | 0.05 | 0.04 | ND |
| 8 | Impurity GR187657X | 1.3 | 0.10 | 0.06 | 0.07 | 0.05 | 0.04 |
| 9 | Total Impurities | | 0.26 | 3.17 | 5.34 | 12.82 | 30.92 |

¹Assay value corresponds to Remifentanil peak
²% Impurity value corresponds to peaks for impurities
³Partial vial of product was used for testing

The results in TABLES 15 through 18 show that the assay of remifentanil for PEG-400/PG formulation at pH 2.0 remained greater than 95% for 7 months stability at 5° C., and the assay of remifentanil for PEG-400/PG formulation at pH 2.0 remained greater than 93% for 7 months stability at 25° C. conditions. The total amount of impurities formed was less than 1.0% for 7-month stability study at 25° C. The assay of remifentanil for PEG-400/PG formulation at pH 2.0 remained greater than 91% for a 7 month stability study at 40° C. The total amount of impurities formed was about 2.6% and the total amount of transesterified degradant was about 1.1% for a 7-month stability study at 40° C. These results demonstrate that remifentanil is stable in PEG-400/PG formulation at pH 2.0. The determined result of 7-month stability at 40° C. in PEG-400/PG formulation has demonstrated that the formulation could have a stability of about 2 years when remifentanil in PEG-400/PG is stored at 5° C. or 25° C. Similar stability results were observed for remifentanil in PEG-400/PG formulation at pH 2.5 and pH 3.0 that was stored at 5° C., 25° C., 40° C., and 60° C. conditions. The observed stability of remifentanil in PEG-400/PG formulation at pH 2.0, 2.5, and 3.0 is similar to the pH stability of remifentanil in reconstituted aqueous solution. The stability of remifentanil in PEG-400/PG at pH values greater than 4.0 was found to be unstable. Based on these results, it is concluded that the stability of remifentanil is optimal at the pH values of 2.0 to pH 3.0 and an acceptable formulation for remifentanil is PEG-400/PG (18%/82%, v/v).

In summary, reformulation of lyophilized formulation of remifentanil, ULTIVA®, as a stable liquid formulation involved the determination of poor stability of remifentanil in aqueous medium due to hydrolysis of the ester group in the molecule. It was found that the rate of hydrolysis increases with increased pH value. To avoid hydrolysis of remifentanil in aqueous medium, non-aqueous solvents were used in the development of liquid formulations. Various non-aqueous solvent systems, such as polyethylene glycol, PEG-400, propylene glycol (PG), mixture of PEG-400/PG, Glycerol, PEG-400/PG (18:82, v/v) with benzyl alcohol and PEG-400/PG (18:82, v/v) with glycine as well as pH adjusted PEG-400/PG mixture, were studied for reformulation development. Based on an acceptable 6-month stability data for storage at 40° C., the formulations containing PEG-400/PG (18:82, v/v) at pH 2.0, pH 2.5, and pH 3.0 were acceptable alternatives to lyophilized formulations of ULTIVA®.

Although various exemplary embodiments have been described in detail with particular reference to certain exemplary aspects thereof, it should be understood that the subject matter disclosed herein is capable of other different embodiments, and its details are capable of modifications in various obvious respects. As is readily apparent to those skilled in the art, variations and modifications can be affected while remaining within the spirit and scope of the disclosure. Accordingly, the foregoing disclosure, description, and figures are for illustrative purposes only, and do not in any way limit the invention, which is defined only by the claims.

1. An injectable liquid pharmaceutical composition comprising remifentanil dissolved in a non-aqueous solvent containing at least one hydrophilic organic component, wherein the composition is water-miscible, has a pH ranging from about 2.0 to about 3.5 and at least one of: i) a room temperature shelf life stability of at least about one year and ii) a refrigerated shelf life stability at about 5° C. of at least about two years.

2. The composition of claim 1 wherein the at least one hydrophilic organic component is a water miscible hydrophilic organic component.

3. The composition of claim 2 wherein the non-aqueous solvent contains at least one of glycol and polyethylene glycol.

4. The composition of claim 3 wherein the non-aqueous solvent contains a mixture of glycol and polyethylene glycol in amounts ranging from about 0.1 volumes to about 10 volumes glycol per volume of polyethylene glycol.

5. The composition of claim 4 wherein the non-aqueous solvent contains a mixture of glycol and polyethylene glycol added in amounts ranging from about 1 volume to about 8 volumes glycol per volume of polyethylene glycol.

6. The composition of claim 5 wherein the non-aqueous solvent contains a mixture of glycol and polyethylene glycol added in amounts ranging from about 2 volumes to about 6 volumes glycol per volume of polyethylene glycol, and the glycol is selected from propylene glycol and glycerol, and the polyethylene glycol is selected from PEG-200, PEG-300, PEG-400 and PEG-500 and PEG-600.

7. The composition of claim 6 wherein the non-aqueous solvent contains a mixture of glycol and polyethylene glycol in amounts ranging from about 3 to about 5 volumes of glycol per volume of polyethylene glycol, the glycol is propylene glycol, and the polyethylene glycol is PEG-400.

8. The composition of claim 1 wherein the non-aqueous solvent contains a mixture of glycol and polyethylene glycol in amounts of about 4 volumes of glycol per volume of polyethylene glycol and the pH of the composition ranges from about 2.0 to about 2.5.

9. The composition of claim 1 which is substantially free of amino acid.

10. The composition of claim 9 which is substantially free of glycine.

11. The composition of claim 1 wherein the remifentanil content after 7 months at 40° C. is greater than about 91% of initial remifentanil content.

12. The composition of claim 1 wherein the remifentanil content after 7 months at 40° C. is greater than about 93% of initial remifentanil content.

13. The composition of claim 1 wherein the remifentanil content after 7 months at 40° C. is greater than about 95% of initial remifentanil content.

14. The composition of claim 1 which comprises no more than about 10 wt. % total impurities for 1 mg doses, no more than about 8 wt. % total impurities for 2 mg doses, and no more than about 6 wt. % total impurities for 5 mg doses.

15. The composition of claim 14 wherein the total impurities include at least one of hydrolysis degradation product GR90291X and heat degradation product GR94219X.

16. The composition of claim 1 which is suitable for parenteral administration in a patient.

17. The composition of claim 1 which is suitable for intravenous administration in a patient.

18. The composition of claim 1 wherein the remifentanil is in the form of a free base.

19. The composition of claim 1 wherein the remifentanil is in the form of a salt.

20. The composition of claim 19 wherein the remifentanil is in the form of a hydrochloride salt.

21. The composition of claim 1 wherein the composition is sterilized.

22. The composition of claim 1 which contains remifentanil hydrochloride in the range of about 0.1 mg/mL to about 5 mg/mL.

23. The composition of claim 1 which contains remifentanil hydrochloride in an amount of about 1 mg/mL.

24. The composition of claim 1 which is provided in a single unit dosage amount selected from 0.25 mg, 0.5 mg, 1 mg, 2 mg, and 5 mg of remifentanil.

25. An infusion mixture which is prepared by adding an aqueous solution to the composition of claim 1 to provide an infusion mixture containing from about 20 to about 250 μg/mL of remifentanil.

26. A method of preparing an infusion mixture which comprises adding an aqueous solution to the composition of claim 1 to provide an infusion mixture containing from about 20 to about 250 μg/mL of remifentanil.

27. A method for preparing an injectable liquid composition of at least one of: i) a room temperature shelf life stability of at least about one year and ii) a refrigerated shelf life stability at about 5° C. of at least about two years, comprising mixing remifentanil with a non-aqueous solvent containing at least one hydrophilic organic component, and adjusting the pH of the resulting mixture to a pH ranging from about 2.0 to about 3.5.

28. The method of claim 27 wherein the mixing comprises sonication.

29. A method of providing analgesia in a mammal comprising administering to such mammal an analgesically effective amount of the composition of claim 1.

30. An injectable liquid pharmaceutical composition comprising remifentanil hydrochloride dissolved in a non-aqueous solvent containing propylene glycol and polyethylene glycol, wherein the composition has a pH ranging from about 2.0 to about 3.5 and at least one of: i) a room temperature shelf life stability of at least about one year and ii) a refrigerated shelf life stability at about 5° C. of at least about two years.

31. The composition of claim 30 which contains remifentanil hydrochloride in an amount ranging from about 0.5 to about 2 mg/mL.

32. The composition of claim 30 which is provided in a single unit dosage amount selected from 0.25 mg, 0.5 mg, 1 mg, 2 mg, and 5 mg of remifentanil.

33. The composition of claim 30 wherein the non-aqueous solvent contains a mixture of propylene glycol and polyethylene glycol in amounts ranging from about 3 to about 5 volumes of propylene glycol per volume of polyethylene glycol, and the polyethylene glycol is PEG-400.

34. The composition of claim 30 wherein the non-aqueous solvent contains a mixture of propylene glycol and polyethylene glycol in amounts of about 4 volumes of propylene glycol per volume of polyethylene glycol, and the pH is in a range of from about 2.0 to about 3.0.

35. An injectable liquid pharmaceutical composition comprising remifentanil dissolved in a non-aqueous solvent containing at least one hydrophilic organic component, wherein the composition is water-miscible, has a pH ranging from about 2.0 to about 3.5 and a shelf life stability at about 40° C. of at least about six months.

36. An injectable liquid pharmaceutical composition comprising remifentanil dissolved in a liquid wherein the composition is characterized by least one of:
i) a shelf life stability at about 40° C. of at least about six months,
ii) a room temperature shelf life stability of at least about one year and,
iii) a refrigerated shelf life stability at about 5° C. of at least about two years.

37. A stable liquid pharmaceutical composition comprising remifentanil in a non-aqueous solvent, in an amount ranging from about 0.25 mg/mL to about 1 mg/mL.

38. The composition of claim 37, wherein remifentanil degrades to an extent of less than 10% in two years.
